(19) United States
(12) Patent Application Publication
(10) Pub. No.: US 2021/0052361 A1
(43) Pub. Date: Feb. 25, 2021

(54) SYSTEM FOR SENSING THE FILLING OF AN EMBOLIC PROTECTION DEVICE BY PRESSURE MONITORING

(71) Applicant: KONINKLIJKE PHILIPS N.V., Eindhoven (NL)

(72) Inventor: Adam TSCHIDA, GREENFIELD, MN (US)

(21) Appl. No.: 16/996,118

(22) Filed: Aug. 18, 2020

Related U.S. Application Data
(60) Provisional application No. 62/890,095, filed on Aug. 22, 2019.

(51) Int. Cl. A61F 2/01; A61B 5/00; A61B 5/021

(52) U.S. Cl. CPC A61F 2/0105; A61B 5/6876; A61B 5/021; A61F 2250/0098; A61B 5/0004; A61F 2002/016; A61B 5/0031

(57) ABSTRACT

An endovascular device including a filter, at least one sensor, and a controller is disclosed. The filter has a proximal end and a distal end, and the filter is inserted into a body lumen and collects embolic materials as the embolic materials travel through the body lumen from the proximal end toward the distal end. The at least one sensor is located near the filter and perform physiological measurements within the body lumen. The controller coupled to the at least one sensor determines a fill level the filter based on the physiological measurements received from the at least one sensor.

### CROSS REFERENCE TO RELATED APPLICATIONS

This application claims the benefit of U.S. Provisional Patent Application No. 62/890,095 filed Aug. 22, 2019. This application is hereby incorporated by reference herein.

### FIELD OF THE DISCLOSURE

The present disclosure generally relates to embolic protection devices. More particularly, the present disclosure relates to sensing when the embolic protection devices are filling up.

### BACKGROUND OF THE DISCLOSURE

Embolic protection and devices therefor are utilized throughout the vasculature in percutaneous peripheral interventions to prevent the potentially fatal passage of embolic material, calcium deposits, and other debris in the bloodstream to smaller vessels where it can obstruct blood flow, as well as from smaller to larger vessels. The dislodgement of embolic material, calcium deposits, and other debris is often associated with procedures which open blood vessels to restore natural blood flow such as stenting, angioplasty, atherectomy, valve replacement or repair, endarterectomy or thrombectomy. Used as an adjunct to these procedures, embolic protection devices trap debris and provide a means for removal from the body.

### SUMMARY OF THE DISCLOSURE

Embodiments of the present disclosure relate to sensing the filling of an embolic protection device by pressure monitoring. Exemplary embodiments include but are not limited to the following examples.

In an exemplary embodiment, an endovascular device comprises: a filter having a proximal end and a distal end, the filter configured to be inserted into a body lumen and collect embolic materials as the embolic materials travel through the body lumen from the proximal end toward the distal end; at least one sensor located near the filter, the at least one sensor configured to perform physiological measurements within the body lumen; and a controller coupled to the at least one sensor, the controller configured to determine, based on the physiological measurements received from the at least one sensor, a fill level for the filter.

In an example of the endovascular device according to the previous paragraph, the endovascular device further comprises a wire extending from the proximal end of the filter to the distal end of the filter, wherein the at least one sensor is arranged on the wire.

In another example of the endovascular device according to any one of the previous paragraphs, the at least one sensor is coupled to the filter and is arranged proximal the proximal end.

In yet another example of the endovascular device according to any one of the previous paragraphs, the at least one sensor is coupled to the filter and is arranged proximal the distal end.

In even another example of the endovascular device according to any one of the previous paragraphs, the at least one sensor is configured to measure blood pressure.

In an example of the endovascular device according to the previous paragraph, the controller determines the fill level of the filter exceeds a threshold when the at least one sensor measures an increase in the blood pressure.

In another example of the endovascular device according to any one of the previous paragraphs, the at least one sensor is configured to measure blood flow.

In an example of the endovascular device according to the previous paragraph, the controller determines the fill level of the filter exceeds a threshold when the at least one sensor measures a decrease in the blood flow.

In yet another example of the endovascular device according to any one of the previous paragraphs, the at least one sensor is a pair of sensors comprising a proximal sensor and a distal sensor, the proximal sensor located proximal the proximal end, and the distal sensor located proximal the distal end.

In an example of the endovascular device according to the previous paragraph, the controller determines whether the fill level of the filter exceeds a threshold based on a difference between the physiologic measurements sensed by the proximal sensor and the physiologic measurements sensed by the distal sensor.

In even another example of the endovascular device according to any one of the previous paragraphs, the filter has one or more characteristics selected from the following group of characteristics: is self-expanding, is a mesh filter, and is radiopaque.

In another example of the endovascular device according to any one of the previous paragraphs, the filter comprises a support structure made of a metal alloy.

In yet another example of the endovascular device according to any one of the previous paragraphs, the filter further comprises a polymer membrane attached to a support structure of the filter.

In an example of the endovascular device according to the previous paragraph, the polymer membrane comprises one or more of the following materials: polyethylene terephthalate (PET), polyvinylidene fluoride (PVDF), perfluoroalkoxy alkane (PFA), fluorinated ethylene propylene (FEP), polytetrafluoroethylene (PTFE), and nylon.

In another exemplary embodiment, a non-transitory computer readable medium storing instructions for execution by one or more processors incorporated into an endovascular system, wherein execution of the instructions by the one or more processors cause the one or more processors to: sense, by at least one sensor located near a filter arranged within a body lumen, physiologic measurements within the body lumen; transmit, by a transmitter coupled to the at least one sensor, the physiologic measurements to a controller; and determine, by the controller, whether a filter of the endovascular device exceeds a threshold fill level based on the physiologic measurements.

In an example of the non-transitory computer readable medium according to the previous paragraph, the at least one sensor is configured to measure blood pressure.

In an example of the non-transitory computer readable medium according to the previous paragraph, the controller determines the fill level of the filter exceeds a threshold when the at least one sensor measures an increase in the blood pressure.

In another example of the non-transitory computer readable medium according to any one of the previous paragraphs, the at least one sensor is configured to measure blood flow.

In an example of the non-transitory computer readable medium according to the previous paragraph, the controller determines the fill level of the filter exceeds a threshold when the at least one sensor measures a decrease in the blood flow.

In yet another example of the non-transitory computer readable medium according to any one of the previous paragraphs, the at least one sensor is a pair of sensors comprising a proximal sensor and a distal sensor, the proximal sensor located proximal a proximal end of the filter, and the distal sensor located proximal a distal end of the filter and wherein the controller is configured to determine the fill level of the filter exceeds a threshold based on a difference between the physiologic measurements sensed by the proximal sensor and the physiologic measurements sensed by the distal sensor.

While multiple embodiments are disclosed, still other embodiments of the present disclosure will become apparent to those skilled in the art from the following detailed description, which shows and describes illustrative embodiments of the present disclosure. Accordingly, the drawings and detailed description are to be regarded as illustrative in nature and not restrictive.

### BRIEF DESCRIPTION OF THE DRAWINGS

The above-mentioned and other features of this disclosure and the manner of obtaining them will become more apparent and the disclosure itself will be better understood by reference to the following description of embodiments of the present disclosure taken in conjunction with the accompanying drawings, wherein:

FIG. 1 is a side view of a catheter for deploying an embolic protection device according to an embodiment disclosed herein;

FIG. 2 is a side view of an embolic protection device according to an embodiment disclosed herein;

FIG. 3 is a side view of another embolic protection device according to an embodiment disclosed herein;

FIG. 4 is a side view of another embolic protection device according to an embodiment disclosed herein;

FIG. 5 is a schematic diagram of an embolic protection system according to an embodiment disclosed herein;

FIG. 6 is a block diagram of a method for determining if the filter is full according to an embodiment disclosed herein.

While the present disclosure is amenable to various modifications and alternative forms, specific embodiments have been shown by way of example in the drawings and are described in detail below. The intention, however, is not to limit the present disclosure to the particular embodiments described. On the contrary, the present disclosure is intended to cover all modifications, equivalents, and alternatives falling within the scope of the present disclosure as defined by the appended claims.

### DETAILED DESCRIPTION OF THE DISCLOSURE

In the following detailed description, reference is made to the accompanying drawings which form a part hereof, and in which is shown by way of illustration specific embodiments in which the present disclosure is practiced. These embodiments are described in sufficient detail to enable those skilled in the art to practice the present disclosure, and it is to be understood that other embodiments can be utilized and that structural changes can be made without departing from the scope of the present disclosure. Therefore, the following detailed description is not to be taken in a limiting sense, and the scope of the present disclosure is defined by the appended claims and their equivalents.

As mentioned above, embolic protection devices trap debris and provide a means for removal from the body. There is a need, however, to monitor if the embolic protection devices are filling up, so that the devices can be taken out when it is sufficiently filled with the embolic material, calcium deposits, and other debris, to prevent such material from overflowing, which may lead to difficulty in safely removing the embolic protection device from the body as well as risks of embolization.

FIG. 1 shows a catheter 500 according to one embodiment, which houses an endovascular device, or more specifically an embolic protection device 100, until the device 100 is deployed at a desired location within the body lumen 2. In one example, the catheter 500 is configured to lead the guidewire 102, filter 110, and the sensors 114, 116 through the body lumen 2 such that the support structure 118 and the filter 110 are in a constricted configuration. While in the constricted configuration, the support structure 118 has a smaller radius than in a fully deployed configuration shown in FIGS. 2-4. During or after deployment, the support structure 118 is then allowed to expand radially to its expanded state, and the filter 110 is also allowed to expand radially and/or axially along the guidewire 102 to collect embolic materials. In one example, the desired location is proximate a thrombus 4 formed on a vessel wall 1 within the lumen 2, such as a location distal to the thrombus 4. As such, when a portion of the thrombus 4 breaks off to form an embolus 3, the filter 110 after being deployed from the catheter 500 can capture and collect the embolus 3, thereby preventing the embolic material from traveling further downstream in the blood vessel 1.

FIG. 2 shows an endovascular device, or more specifically an embolic protection device 100, according to one embodiment. The device 100 is located within a lumen 2 of a vessel 1 in order to capture any embolus 3 which may be located within the lumen 2. The device 100 includes a guidewire 102 with a proximal end 104, a distal end 106, and an intermediate portion 108 extending between the two ends, where the words “proximal” and “distal” are with respect to the direction in which the blood flows within the vessel lumen 2. In this example, blood flows within the vessel lumen 2 from the proximal end 104 to the distal end 106, such that when there are emboli 3 within the vessel, the emboli 3 flow from the proximal end 104 to the distal end 106 of the device 100. The device 100 also has a filter 110 located between the proximal end 104 and the distal end 106 with an opening 112 facing the proximal end 104. The filter 110 is configured to capture and collect the embolic materials 3 as they travel through the lumen 2. In some examples, the filter 110 is a self-expanding filter such that as the device 100 is deployed in the lumen 2, the filter 110 is able to expand on its own. In other examples, the filter 110 is expanded manually or mechanically. In some examples, the filter 110 is a mesh filter so that the filter 110 does not obstruct the lumen 2 so much that the blood flow is compromised. In some examples, the filter 110 is radiopaque such that the location of the filter 110 can be detected using E-rays or similar radiation.

The device 100 further includes a first sensor 114 located on the guidewire 102 between the proximal end 104 and the filter 110, and a second sensor 116 located on the distal end 106 of the guidewire 102. In some examples, the second sensor 116 is located on the guidewire 102 between the distal end 106 and the filter 110. The two sensors 114, 116 are located near the filter 110 and collectively measure physiologic measurements within the lumen 2. In one example, the sensors 114, 116 are intra-arterial blood pressure (IBP) monitoring devices that measure blood pressure in their respective locations such that the first sensor 114 measures the blood pressure within the lumen 2 before contacting the filter 110, and the second sensor 116 measures the blood pressure within the lumen 2 after passing through the filter 110. The presence of embolic materials 3 collected within the filter 110 affects the amount of blood that can pass through the filter, therefore when there is an abundant amount of embolic materials 3, the pressure measured by the first sensor 114 should be considerably higher than that measured by the second sensor 116. In another example, the sensors 114, 116 are pressure wires as commonly used in fractional flow reserve (FFR) and iFR (instantaneous wave-free ratio) procedures. In another example, the sensors 114, 116 are embedded into the respective portions of the guidewire 102.

The sensors 114, 116 detect and send corresponding signals indicative of the fill level of the filter 110, such as whether the filter 110 is partially or completely full. In one example, the sensors 114, 116 detect and measure the blood pressure (and/or blood flow), and the controller 401 calculates the differential blood pressure between the sensors 114, 116. The controller 402 has a plurality of differential blood pressure thresholds that correspond to a predetermined number of fill levels. In one example, the thresholds are indicative of the filter 110 being 25%, 50%, 75%, or 100% full, or any other set of suitable threshold fill levels, so the physician can decide at which point to replace the filter. In another example, the sensors 114, 116 detect when the filter 110 is filled to a specific percentage threshold associated with one or more different recovery catheter sizes, so the physician can decide the size of the recovery catheter that should be used to retrieve the filter from the inside the patient. In one example, the blood pressure measurement that is used for determining whether the filter is filled is based on the mean arterial pressure calculated from the measured systolic and diastolic pressure readings. In another example, the systolic pressure is used as the pressure measurements, and in yet another example, the diastolic pressure is used instead.

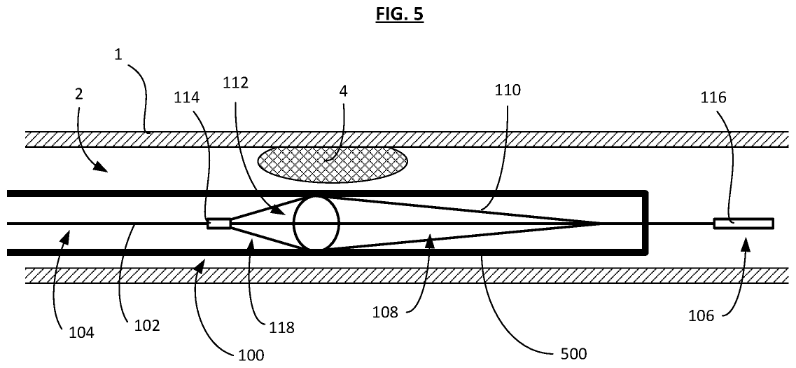

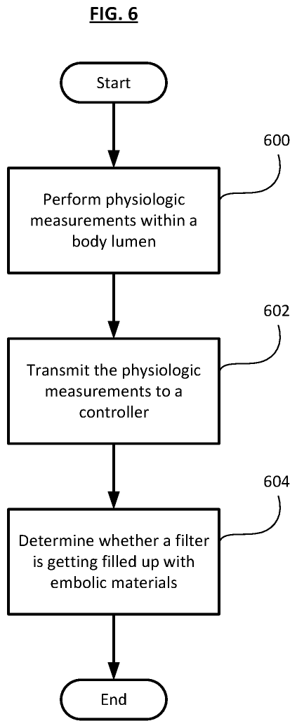

In some examples, the first sensor 114 is smaller in size than the second sensor 116. This is because the amount of obstruction in the proximal side of the filter 110 is preferably minimized to prevent accumulation of the embolic material 3 on the first sensor 114. Therefore, in one example, the second sensor 116 also includes a transmitter (for example, transmitter 406 shown in FIG. 5, explained below) attached thereto which enables the measurement data taken by both the first and second sensors 114, 116 to be sent to a remove device such as a controller 402 or processing unit for analysis (shown in FIG. 4). In another example, the second sensor 116 also includes a battery (not shown) coupled to the transmitter such that the transmitter 406 can send measurement data to the controller 402 at a predetermined interval. Without the battery, the transmitter 406 may be a passive transmitter that is activated only when an inductive power source (not shown) is placed at a proximity, but when the battery is coupled to the transmitter 406, the transmitter 406 can actively transmit sensor data in the absence of such inductive power source. Advantages in having the transmitter attached to the second sensor 116 includes a reduction in the size of the first sensor 114.

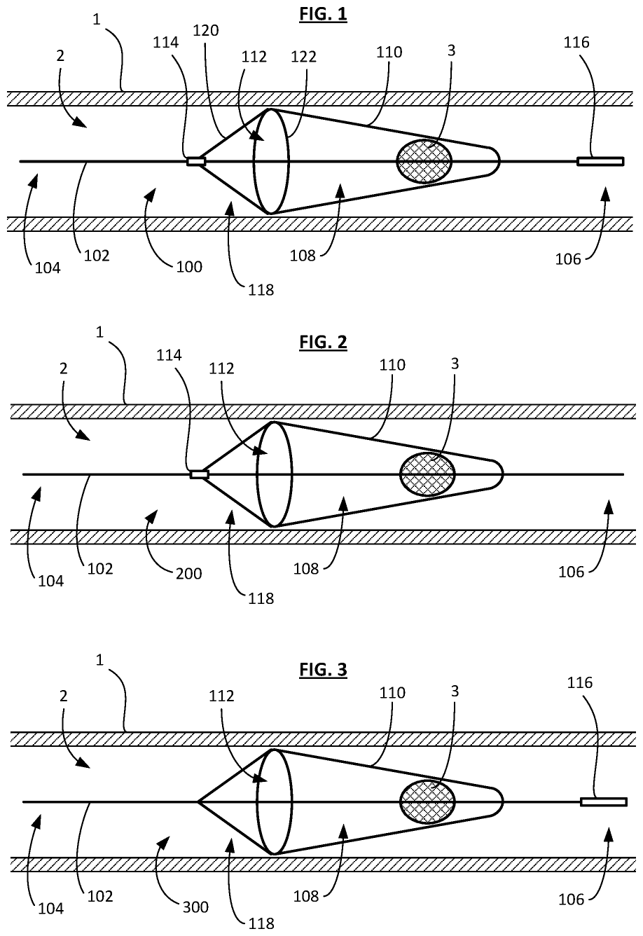

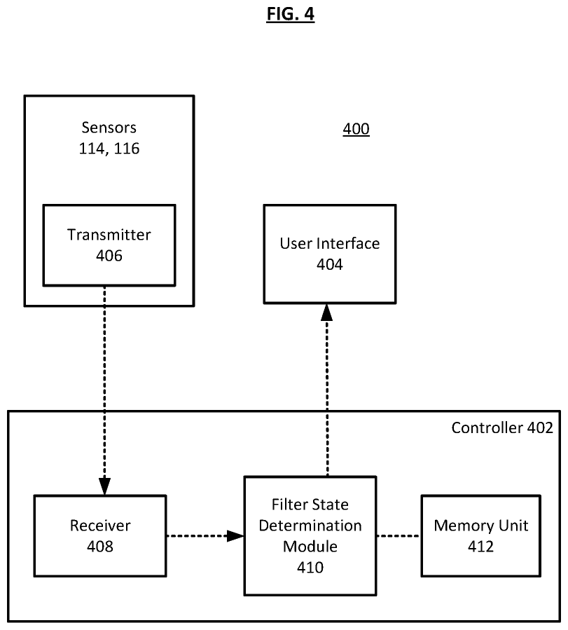

FIGS. 3 and 4 show other embodiments of the embolic protection device when there is only one sensor instead of two as shown in FIG. 2. In FIG. 3, an embolic protection device 200 according to one example has the sensor 114 attached to the guidewire 102 between the proximal end 104 and the filter 110. In this example, the sensor 114 can be a blood pressure measurement device as explained above, but instead of the ratio or differential between the two sensors, the sensor 114 measures any change in pressure readings in the portion of the lumen 2 between the proximal end 104 and the filter 110. In one example, the blood pressure is measured by the sensor 114 over multiple time windows, and for each time window, an average blood pressure value is calculated and recorded. When the average blood pressure measured during a later time window is higher than the average blood pressure measured during an earlier time window, this increase in blood pressure indicates that the filter 110 is being filled with embolic materials 3. Similar to the example shown in FIG. 2, the sensors 114, 116 can detect when the filter 110 is past any set of threshold fill levels so the physician can decide when to replace the filter as well as what size of catheter to use to retrieve the filter.

In one example, the sensor 114 is a flowmeter which measures the blood flow passing by or through the sensor 114. When the filter 110 is relatively filled with embolic materials 3, the rate at which blood flows through the lumen 2 near the proximal side of the filter 110 decreases because of the blockage caused by the embolic materials 3 within the filter 110. As such, the rate of blood flow can be measured and recorded such that when a decrease in the blood flow is detected, it would indicate that the filter 110 is being filled with embolic materials 3. The same measurement techniques explained above can be employed for an embolic protection device 300 according to one example shown in FIG. 4, where the device 300 has the sensor 116 attached to the guidewire 102 on the distal end 106, or between the distal end 106 and the filter 110 in some examples, as appropriate. For example, the sensors 114, 116 detect and measure the blood flow, and the controller 401 calculates the differential blood flow between the sensors 114, 116. The controller 402 has a plurality of differential blood flow thresholds that correspond to a predetermined number of fill levels. In one example, the thresholds are indicative of the filter 110 being 25%, 50%, 75%, or 100% full, or any other set of suitable threshold fill levels, so the physician can decide at which point to replace the filter. In another example, the sensors 114, 116 detect when the filter 110 is filled to a specific percentage threshold associated with one or more different recovery catheter sizes, so the physician can decide the size of the recovery catheter that should be used to retrieve the filter from the inside the patient.

In another example, the sensor 114′ is a dual pressure and flowmeter which measures the blood pressure and blood flow passing by or through the sensor 114′, respectively, and the sensor 116′ is a dual pressure and flowmeter which measures the blood pressure and blood flow passing by or through the sensor 116′, respectively. The controller 401 is also capable of detecting the changes such as an increase and/or decrease in the blood pressure and/or blood flow from the sensors 114′, 116′. The combination of using both the blood pressure and blood flow signals may provide a more accurate determination of the fill level for the filter 110. In some examples, the sensor 114′ comprises more than one sensor such that a first sensor of the sensors 114′ measures blood pressure and a second sensor of the sensor 114′ measures blood flow. Additionally, or alternatively, the sensor 116′ comprises more than one sensor such that a first sensor of the sensors 116′ measures blood pressure and a second sensor of the sensor 116′ measures blood flow.

In the above examples, the sensors 114, 116 can be located within a short distance from the filter 110 such that changes in sensor measurements are immediately detected. In one example, the sensors 114, 116 can be located approximately 1 mm, 5 mm, 10 mm, 15 mm, 20 mm, 25 mm, 30 mm, or any distance therebetween from the filter 110, as appropriate. In some examples, the filter 110 is a polymer membrane supported by a support structure 118, as shown in FIGS. 2-4, which includes support members 120 extending radially from the guidewire 102 and forming a ring 122 which defines the opening 112. The number of support members 120 can be one or more, as appropriate. The ring 122 can be circular or ovular, or in some examples replaced by a polygonal structure, as appropriate. In some examples, the support structure 118 is made of a metal alloy such as nickel-titanium alloys, stainless steel, cobalt chrome, other shape memory alloys, or other metal alloys. The polymer membrane can be made of any suitable polymer material such as, for example, polyethylene terephthalate (PET), polyvinylidene fluoride (PVDF), perfluoroalkoxy alkane (PFA), fluorinated ethylene propylene (FEP), polytetrafluoroethylene (PTFE), nylon, and other suitable polymers. In some examples, the support structure 118 is made of one or more suitable polymer materials listed above.

FIG. 5 shows an embolic protection system 400 using the sensors 114, 116, a controller 402, and a user interface 404 according to one embodiment. The sensors 114, 116 use a transmitter 406 to transmit the measurement data to a receiver 408 located in the controller 402. The data is then transmitted to a filter state determination module 410 which uses the measurement data to determine whether the filter 110 is full or close to being full. In one example, the controller 402 is an extracorporeal device which wirelessly receives the sensor data, and the controller 402 may be any suitable processing unit such as a central processing unit (CPU), a system-on-a-chip (SoC), etc. The filter state determination module 410 may be any suitable processing unit coupled with one or more suitable memory unit 412 such as random-access memory (RAM), flash memory, etc., capable of storing the instructions necessary to analyze the sensor data as well as any previous measurement data transmitted from the sensors 114, 116. After the analysis, the filter state determination module 410 sends a command signal to a user interface 404, which includes a display to show any notification from the system, to display a suitable message according to the analysis. For example, if the module 410 determines that the filter 110 is almost full and needs to be replaced, the user interface 404 should appropriately display a message indicating for a physician to replace the filter 110 to prevent the filter 110 from obstructing blood flow within the body lumen 2. In another example, the filter 110 can be taken out, cleaned, and then placed back into the patient's body.

FIG. 6 shows a method of determining if the filter 110 is filled with embolic materials 3 according to an embodiment. In block 600, physiologic measurements are performed with the body lumen 2 by one or both of the sensors 114, 116. The physiologic measurements may be pressure or flow measurements. In block 602, these measurements are transmitted to the controller 402. The controller 402 then uses the measurement data to determine whether the filter 110 is reaching a predetermined threshold fill level. According to block 604, the determination depends on whether blood pressure and/or blood flow is measured. If the blood pressure is measured, in block 606, the controller 402 determines that the filter 110 exceeds the threshold fill level when the sensors 114, 116 measure an increase in blood pressure. Otherwise, if the blood pressure and/or the blood flow is measured, in block 608, the controller 402 determines that the filter exceeds the corresponding threshold fill level when the sensors 114, 116 measure a predetermined decrease in blood pressure and/or blood flow.

It is to be understood that the above description is intended to be illustrative, and not restrictive. Many other embodiments will be apparent to those of skill in the art upon reading and understanding the above description. For example, it is contemplated that features described in association with one embodiment are optionally employed in addition or as an alternative to features described in associate with another embodiment. The scope of the present disclosure should, therefore, be determined with reference to the appended claims, along with the full scope of equivalents to which such claims are entitled.

What is claimed is:

1. An endovascular device comprising:
a filter having a proximal end and a distal end, the filter configured to be inserted into a body lumen and collect embolic materials as the embolic materials travel through the body lumen from the proximal end toward the distal end;
at least one sensor located near the filter, the at least one sensor configured to perform physiological measurements within the body lumen; and
a controller coupled to the at least one sensor, the controller configured to determine, based on the physiological measurements received from the at least one sensor, a fill level for the filter.

2. The endovascular device of claim 1 further comprising a wire extending from the proximal end of the filter to the distal end of the filter, wherein the at least one sensor is arranged on the wire.

3. The endovascular device of claim 1, wherein the at least one sensor is coupled to the filter and is arranged proximal the proximal end.

4. The endovascular device of claim 1, wherein the at least one sensor is coupled to the filter and is arranged proximal the distal end.

5. The endovascular device of claim 1, wherein the at least one sensor is configured to measure blood pressure.

6. The endovascular device of claim 5, wherein the controller determines the fill level of the filter exceeds a threshold when the at least one sensor measures an increase in the blood pressure.

7. The endovascular device of claim 1, wherein the at least one sensor is configured to measure blood flow.

8. The endovascular device of claim 7, wherein the controller determines the fill level of the filter exceeds a threshold when the at least one sensor measures a decrease in the blood flow.

9. The endovascular device of claim 1, wherein the at least one sensor is a pair of sensors comprising a proximal sensor and a distal sensor, the proximal sensor located proximal the proximal end, and the distal sensor located proximal the distal end.

10. The endovascular device of claim 9, wherein the controller determines whether the fill level of the filter exceeds a threshold based on a difference between the physiologic measurements sensed by the proximal sensor and the physiologic measurements sensed by the distal sensor.

11. The endovascular device of claim 1, wherein the filter has one or more characteristics selected from the following group of characteristics: is self-expanding, is a mesh filter, and is radiopaque.

12. The endovascular device of claim 1, wherein the filter comprises a support structure made of a metal alloy.

13. The endovascular device of claim 1, wherein the filter further comprises a polymer membrane attached to a support structure of the filter.

14. The endovascular device of claim 13, wherein the polymer membrane comprises one or more of the following materials: polyethylene terephthalate (PET), polyvinylidene fluoride (PVDF), perfluoroalkoxy alkane (PFA), fluorinated ethylene propylene (FEP), polytetrafluoroethylene (PTFE), and nylon.

15. A non-transitory computer readable medium storing instructions for execution by one or more processors incorporated into an endovascular system, wherein execution of the instructions by the one or more processors cause the one or more processors to:
sense, by at least one sensor located near a filter arranged within a body lumen, physiologic measurements within the body lumen;
transmit, by a transmitter coupled to the at least one sensor, the physiologic measurements to a controller; and
determine, by the controller, whether a filter of the endovascular device exceeds a threshold fill level based on the physiologic measurements.

16. The non-transitory computer readable medium of claim 15, wherein the at least one sensor is configured to measure blood pressure.

17. The non-transitory computer readable medium of claim 16, wherein the controller determines the fill level of the filter exceeds a threshold when the at least one sensor measures an increase in the blood pressure.

18. The non-transitory computer readable medium of claim 15, wherein the at least one sensor is configured to measure blood flow.

19. The non-transitory computer readable medium of claim 18, wherein the controller determines the fill level of the filter exceeds a threshold when the at least one sensor measures a decrease in the blood flow.

20. The non-transitory computer readable medium of claim 15, wherein the at least one sensor is a pair of sensors comprising a proximal sensor and a distal sensor, the proximal sensor located proximal a proximal end of the filter, and the distal sensor located proximal a distal end of the filter and wherein the controller is configured to determine the fill level of the filter exceeds a threshold based on a difference between the physiologic measurements sensed by the proximal sensor and the physiologic measurements sensed by the distal sensor.
